(19) United States
(12) Patent Application Publication
(10) Pub. No.: US 2024/0050034 A1
(43) Pub. Date: Feb. 15, 2024

(54) SYSTEMS AND METHODS FOR MONITORING AND TRACKING SWELLING

(71) Applicant: KCI Manufacturing Unlimited Company, Westmeath (IE)

(72) Inventors: Jonathan G. REHBEIN, San Antonio, TX (US); Luke PERKINS, San Antonio, TX (US); David MERCER, San Antonio, TX (US)

(73) Assignee: KCI Manufacturing Unlimited Company, Westmeath (IE)

(21) Appl. No.: 18/271,114

(22) PCT Filed: Dec. 8, 2021
(86) PCT No.: PCT/IB2021/061446

§ 371 Date: Jul. 6, 2023

Related U.S. Application Data
(60) Provisional application No. 63/134,685, filed on Jan. 7, 2021.

(51) Int. Cl. A61B 5/00; A61B 5/107

(52) U.S. Cl. CPC A61B 5/4878; A61B 5/6802; A61B 5/0077; A61B 5/6898; A61B 5/1079; A61B 5/746; A61B 5/0002; A61B 2560/0223; A61B 5/743

(57) ABSTRACT

A system for monitoring and tracking swelling of a patient's limb includes a dressing, an imaging device, and processing circuitry. The dressing is positioned on a patient's limb over a wound and includes a pattern indicator. The imaging device is configured to obtain an image of the dressing and the pattern indicator. The processing circuitry is configured to obtain an initial image of the pattern indicator, obtain one or more subsequent images of the pattern indicator at later times, and determine a swelling change of the patient's limb using the initial image and the one or more subsequent images.

### CROSS-REFERENCE TO RELATED APPLICATIONS

This application is a U.S. National Stage entry of International Patent Application No. PCT/IB2021/061336, filed on Dec. 8, 2021, which claims the benefit of priority to U.S. Provisional Application No. 63/134,685, filed on Jan. 7, 2021, which is incorporated herein by reference in its entirety.

### BACKGROUND

The present disclosure relates to swelling of a patient's limb. More particularly, the present disclosure relates to tracking and monitoring swelling increases or decreases of the patient's limb.

### SUMMARY

One implementation of the present disclosure is a system for monitoring and tracking swelling of a patient's limb, according to some embodiments. The system can include a dressing, an imaging device, and processing circuitry. The dressing is positioned on a patient's limb over a wound and includes a pattern indicator. The imaging device is configured to obtain an image of the dressing and the pattern indicator. The processing circuitry is configured to obtain an initial image of the pattern indicator, obtain one or more subsequent images of the pattern indicator at later times, and determine a swelling change of the patient's limb using the initial image and the one or more subsequent images.

In some embodiments, the processing circuitry and the imaging device are components of a smartphone.

In some embodiments, the processing circuitry is configured to perform an image analysis technique to determine a baseline dimension of the pattern indicator using the initial image of the pattern indicator. In some embodiments, the processing circuitry is configured to perform the image analysis technique to determine one or more subsequent dimensions of the pattern indicator using the one or more subsequent images of the pattern indicator obtained at later times. In some embodiments, the processing circuitry is configured to determine relative increase or decrease amounts between the baseline dimension of the pattern indicator and the one or more subsequent dimensions of the pattern indicator.

In some embodiments, the processing circuitry is configured to determine a relative increase or decrease in at least one of a circumference, diameter, or volume of the patient's limb using the relative increase or decrease amounts.

In some embodiments, the pattern indicator includes a grid and a reference indicator. The grid may be configured to expand or contract with swelling increases or decreases of the patient's limb. The reference indicator may be fixedly coupled with the grid. The reference indicator can be a rigid member that does not expand or contract with swelling increases or decreases of the patient's limb.

In some embodiments, the processing circuitry is configured to calibrate any of the initial image or the one or more subsequent images using image data of the reference indicator to account for a relative orientation or position between the imaging device and the pattern indicator.

In some embodiments, the processing circuitry is configured to generate progress data of multiple values of the swelling change of the patient's limb using the initial image and multiple subsequent images. The processing circuitry can generate a progress report using the progress data and provide the progress report to at least one of the patient via a display screen or a healthcare professional via a remote system.

In some embodiments, the progress report includes at least one of a current value of a circumference, diameter, or volume of the patient's limb, a current value of a dimension of the pattern indicator, historical values of the circumference, diameter, or volume of the patient's limb, historical values of the dimension of the pattern indicator, or a graph showing swelling changes of the patient's limb over time.

In some embodiments, the system further includes a display screen. The processing circuitry can be configured to periodically prompt the patient to capture a new image of the pattern indicator via the display screen.

In some embodiments, the processing circuitry is configured to alert the patient or a healthcare professional in response to the swelling increasing by a predetermined amount or more.

In some embodiments, the system further includes a negative pressure wound therapy (NPWT) unit and a tubular member. The NPWT unit can be configured to draw a negative pressure within the dressing through the tubular member.

Another implementation of the present disclosure is a controller for monitoring and tracking swelling of a patient's limb, according to some embodiments. The controller includes processing circuitry configured to obtain an initial image of a pattern indicator positioned at the patient's limb using an imaging device. The processing circuitry can be configured to obtain one or more subsequent images of the pattern indicator at later times using the imaging device. The processing circuitry can also be configured to determine a swelling change of the patient's limb using the initial image and the one or more subsequent images. The processing circuitry can also be configured to provide a report to at least one of the patient or a healthcare provider including the swelling change of the patient's limb.

In some embodiments, the controller is a patient's smartphone with a mobile application installed on the processing circuitry. The processing circuitry and the imaging device may be components of the patient's smartphone.

In some embodiments, the processing circuitry is configured to perform an image analysis technique to determine a baseline dimension of the pattern indicator using the initial image of the pattern indicator. The processing circuitry can also be configured to perform the image analysis technique to determine one or more subsequent dimensions of the pattern indicator using the one or more subsequent images of the pattern indicator obtained at later times. The processing circuitry can also be configured to determine relative increase or decrease amounts between the baseline dimension of the pattern indicator and the one or more subsequent dimensions of the pattern indicator.

In some embodiments, the processing circuitry is configured to determine a relative increase or decrease in at least one of a circumference, diameter, or volume of the patient's limb using the relative increase or decrease amounts.

In some embodiments, the pattern indicator includes a grid configured to expand or contract with swelling increases or decreases of the patient's limb. The pattern indicator can also include a reference indicator fixedly coupled with the grid. The reference indicator may be a rigid member that does not expand or contract with swelling increases or decreases of the patient's limb.

In some embodiments, the processing circuitry is configured to calibrate any of the initial image or the one or more subsequent images using image data of the reference indicator to account for a relative orientation or position between the imaging device and the pattern indicator.

In some embodiments, the processing circuitry is configured to generate progress data of multiple values of the swelling change of the patient's limb using the initial image and multiple subsequent images. The processing circuitry can also be configured to generate a progress report using the progress data and provide the progress report to at least one of the patient via a display screen or a healthcare professional via a remote system.

In some embodiments, the progress report includes at least one of a current value of a circumference, diameter, or volume of the patient's limb, a current value of a dimension of the pattern indicator, historical values of the circumference, diameter, or volume of the patient's limb, historical values of the dimension of the pattern indicator, or a graph showing swelling changes of the patient's limb over time.

In some embodiments, the processing circuitry is configured to periodically prompt the patient to capture a new image of the pattern indicator via a display screen.

In some embodiments, the processing circuitry is configured to alert the patient or a healthcare professional in response to the swelling increasing by a predetermined amount or more.

Another implementation of the present disclosure is a method for monitoring and tracking swelling of a patient's limb, according to some embodiments. The method can include obtaining an initial image of a pattern indicator positioned at the patient's limb using an imaging device. The method can also include obtaining one or more subsequent images of the pattern indicator at later times using the imaging device. The method can also include determining a swelling change of the patient's limb using the initial image and the one or more subsequent images. The method can also include providing a report to at least one of the patient or a healthcare provider including the swelling change of the patient's limb.

In some embodiments, the method further includes performing an image analysis technique to determine a baseline dimension of the pattern indicator using the initial image of the pattern indicator. The method can also include performing the image analysis technique to determine one or more subsequent dimensions of the pattern indicator using the one or more subsequent images of the pattern indicator obtained at later times. The method can also include determining relative increase or decrease amounts between the baseline dimension of the pattern indicator and the one or more subsequent dimensions of the pattern indicator.

In some embodiments, the method further includes determining a relative increase or decrease in at least one of a circumference, diameter, or volume of the patient's limb using the relative increase or decrease amounts.

In some embodiments, pattern indicator includes a grid and a reference indicator. The grid can be configured to expand or contract with swelling increases or decreases of the patient's limb. The reference indicator can be fixedly coupled with the grid. The reference indicator can be a rigid member that does not expand or contract with swelling increases or decreases of the patient's limb.

In some embodiments, the method further includes calibrating any of the initial image or the one or more subsequent images using image data of the reference indicator to account for a relative orientation or position between the imaging device and the pattern indicator.

In some embodiments, the method further includes generating progress data of multiple of the swelling change of the patient's limb using the initial image and multiple subsequent images. The method can also include generating a progress report using the progress data and providing the progress report to at least one of the patient via a display screen or a healthcare professional via a remote system.

In some embodiments, the progress report includes at least one of a current value of a circumference, diameter, or volume of the patient's limb, a current value of a dimension of the pattern indicator, historical values of the circumference, diameter, or volume of the patient's limb, historical values of the dimension of the pattern indicator, or a graph showing swelling changes of the patient's limb over time.

In some embodiments, the method further includes periodically prompting the patient to capture a new image of the pattern indicator via a display screen.

In some embodiments, the method further includes alerting the patient or a healthcare professional in response to the swelling increasing by a predetermined amount or more.

Those skilled in the art will appreciate that the summary is illustrative only and is not intended to be in any way limiting. Other aspects, inventive features, and advantages of the devices and/or processes described herein, as defined solely by the claims, will become apparent in the detailed description set forth herein and taken in conjunction with the accompanying drawings.

### BRIEF DESCRIPTION OF THE DRAWINGS

The disclosure will become more fully understood from the following detailed description, taken in conjunction with the accompanying figures, wherein like reference numerals refer to like elements, in which:

FIG. 1 is a diagram of a system for automatically monitoring and tracking swelling or healing progression of a patient's limb, according to some embodiments;

FIG. 2 is a diagram of a pattern indicator in a rest or un-deformed state, according to some embodiments;

FIG. 3 is a diagram of the pattern indicator of FIG. 2 after experiencing an amount of deformation or contraction, according to some embodiments;

FIG. 4A is a diagram of a pattern indicator in a rest or un-deformed state, according to some embodiments;

FIG. 4B is a diagram of the pattern indicator of FIG. 4A showing the pattern indicator after having undergone a first amount of contraction, according to some embodiments;

FIG. 4C is a diagram of the pattern indicator of FIG. 4B showing the pattern indicator after having undergone an additional amount of contraction relative, according to some embodiments;

FIG. 5A is a diagram of a pattern indicator in a rest or un-deformed state, according to some embodiments;

FIG. 5B is a diagram of the pattern indicator of FIG. 5A after having undergone a first amount of expansion, according to some embodiments;

FIG. 5C is a diagram of the pattern indicator of FIG. 5B after having undergone an additional amount of expansion, according to some embodiments;

FIG. 6 is a front view of a smartphone showing a progress report of swelling reduction of a patient's limb, according to some embodiments;

FIG. 7 is a front view of a smartphone showing another progress report of swelling reduction of a patient's limb, according to some embodiments;

FIG. 8 is a block diagram of the system of FIG. 1, showing a smartphone or controller of the system in greater detail, according to some embodiments

FIG. 9 is a flow diagram of a process for automatically monitoring and tracking healing progression or swelling progression of a patient's limb, according to some embodiments;

FIG. 10 is a graph showing a dimension of a patient's limb that indicates a degree of swelling decreasing over time, according to some embodiments;

FIG. 11 is a graph showing a dimension of a patient's limb that indicates a degree of swelling increasing at a particular point in time, according to some embodiments; and

FIG. 12 is an example healing or swelling progress report of a patient's limb that can be provided to a patient via a display screen of a personal computer device or can be provided to healthcare professional, according to some embodiments.

### DETAILED DESCRIPTION

Before turning to the FIGURES, which illustrate the exemplary embodiments in detail, it should be understood that the present application is not limited to the details or methodology set forth in the description or illustrated in the FIGURES. It should also be understood that the terminology is for the purpose of description only and should not be regarded as limiting.

### Overview

Referring generally to the FIGURES, systems and methods for monitoring and tracking swelling or healing of a patient's limb or body part are shown. The systems and methods described herein can be performed using a patient's smartphone that is loaded with a mobile application to configure the patient's smartphone to perform the functionality described herein. The system can also include a dressing and a therapy system configured to draw a negative or vacuum pressure at a wound over which the dressing is positioned to facilitate or improve healing of the wound. The dressing may include a pre-placed grid or other pattern indicator that is positioned on the dressing or underneath an outer layer of the dressing. The grid may be viewable from an exterior of the dressing. The system can also include an imaging device (e.g., a camera of the smartphone) that can be directed towards (e.g., orthogonally) the dressing to capture an image of the grid. An initial image of the grid can be captured. Subsequent images of the dressing and the grid can be captured at a later time (e.g., periodically over a future time period). The smartphone may perform an image analysis technique to compare the subsequent images to the initial image, or to a previously obtained image. The image analysis technique can use deformations (e.g., expansions or contractions) of the grid to determine an increase or decrease amount in swelling (e.g., changes in diameter of the patient's limb, changes in circumference of the patient's limb, etc.). Such increases or decreases in swelling can be recorded and tracked over time and used by the smartphone to generate a progress report, which can be provided to the patient (e.g., via a display screen of the smartphone) or can be provided to a healthcare professional (e.g., via a remote system).

The systems and methods described herein can also facilitate determining alerts or warnings in response to swelling increases. For example, if an estimated circumference or diameter or volume of the patient's limb increases by a threshold amount or more (e.g., as set by a healthcare provider), the smartphone may provide an alert to the patient and/or to a healthcare professional via a remote system. The healthcare professional may then schedule a time for an in-person visit with the patient.

The systems and methods described herein can additional be used to monitor or predict swelling progression for surgical scheduling purposes. For example, the smartphone may track swelling reduction over time, determine a trend (e.g., perform a regression technique to obtain an equation of a linear trendline) and predict a time at which swelling will be sufficiently low to perform surgery. In some embodiments, the smartphone provides the trend to a remote healthcare system which can use the trend to determine a time at which swelling will be sufficiently low or reduced to perform a surgery.

It should be understood that while the systems and methods described herein are related to swelling applications of a patient's limb, the systems and methods can also be applied to other body parts. For example, the grid or the pattern indicator can also be provided to a patient's stomach or waistline to track bloating increases or decreases, weight loss, muscle changes, etc.

### System

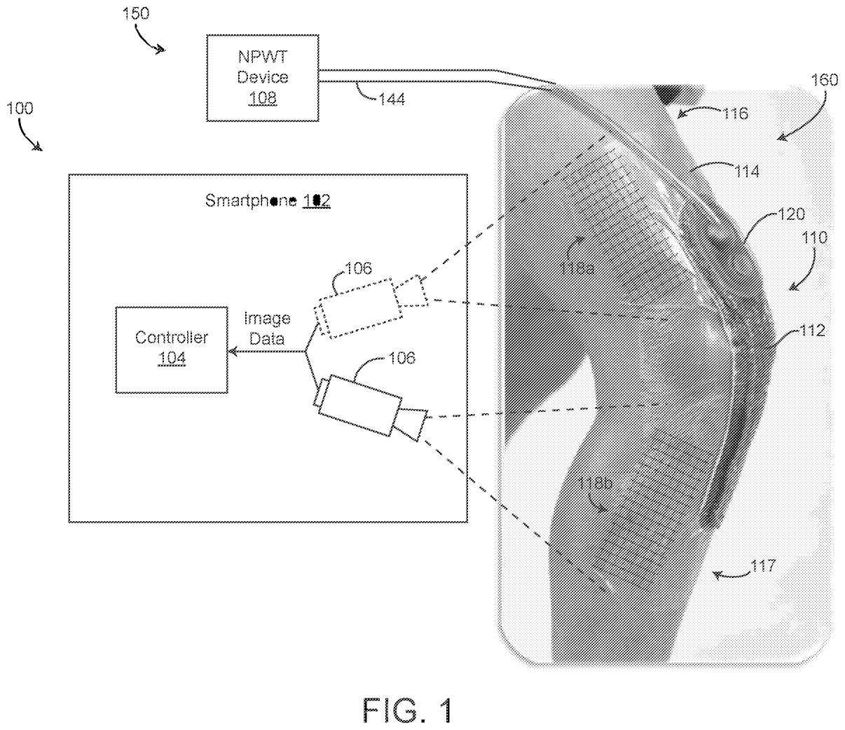

Referring particularly to FIG. 1, a system 100 for automatically obtaining, monitoring, and/or tracking swelling changes of a patient's limb is shown, according to some embodiments. The system 100 includes an image capture and/or processing device, shown as smartphone 102, and a negative pressure wound therapy (NPWT) system 150 that is configured to provide NPWT to a patient's limb 160. The NPWT system 150 can include a NPWT device 108 that is configured to provide NPWT to the patient's limb 160 through a tubular member 144 and a dressing 110. The dressing 110 can be positioned at a wound site of the patient's limb 160 and may be configured to define a sealed inner volume.

The dressing 110 can include a dressing layer 112 (e.g., a foam layer, a set of different foam layers, absorbing layers, etc.), a wound drape 114, and a coupler 120. The wound drape 114 may cover the dressing layer 112 and define the sealed inner volume of the dressing 110. The coupler 120 facilitates fluidly coupling the sealed inner volume of the dressing 110 with the NPWT device 108 through the tubular member 144. The NPWT device 108 can include a pump configured to draw a negative or vacuum pressure at the wound site of the patient's limb 160 through the tubular member 144 to facilitate improved healing and to remove wound exudate from the wound site (e.g., through the tubular member 144).

When a patient undergoes NPWT, or as a patient's wound heals, swelling may reduce or increase, depending on healing progression. For example, for a wound at a patient's knee, a patient's upper limb 116 may reduce or increase in diameter and/or circumference due to swelling. Similarly a patient's lower limb 117 may reduce or increase in diameter and/or circumference due to swelling as the wound healing progresses or deteriorates. Advantageously, the systems and methods described herein can be used to automatically monitor, track, and report swelling reductions or increases, thereby monitoring healing progression of the wound. For example, in the application where the patient has a wound at their knee, it may be desirable to monitor and track swelling at the patient's upper limb 116 and at the patient's lower limb 117.

The dressing 110 can include one or more reference points, reference indicators, printed grids, patterns, visual indicators, colored patterns, lines, etc., shown as pattern indicators 118. The pattern indicators 118 may be printed onto the wound drape 114, the dressing 110, the dressing layer 112, etc. In some embodiments, the pattern indicators 118 are positioned on a portion of the dressing 110 that is configured to deform (e.g., elastically stretch, etc.) as diameter and/or circumference of the patient's limb 160 changes over time (e.g., due to increased or decreased swelling). In this way, the pattern indicators 118 can deform as the diameter and/or circumference (or other dimension) of the patient's limb 160 decreases due to reduced swelling (or increases due to increased swelling).

The pattern indicators 118 can be positioned proximate or at a portion of the patient's limb 160 for which swelling monitoring and tracking is desired. For example, the pattern indicators 118 may be positioned at the patient's upper limb 116 (e.g., shown as a first pattern indicator 118a) and at the patient's lower limb 117 (e.g., shown as a second pattern indicator 118b) so that swelling at the patient's upper limb 116 and lower limb 117 can be tracked and monitored over time.

Referring still to FIG. 1, the system 100 includes an imaging device 106, according to some embodiments. The imaging device 106 can be a camera or any other device configured to capture image data. The imaging device 106 may be a component of the smartphone 102. The imaging device 106 may be directed or pointed towards either of or both of the pattern indicators 118 so that an image can be captured of the pattern indicators 118. For example, the smartphone 102 can be a patient's smartphone so that the patient may obtain images of the pattern indicators 118 themselves. The smartphone 102 and the imaging device 106 can be directed towards the first pattern indicator 118a so that an image can be obtained of the first pattern indicator 118a, and then directed towards the second pattern indicator 118b so that an image can be obtained of the second pattern indicator 118b. The images can be provided to a controller 104 of the smartphone 102 as image data.

### Pattern Indicator Examples

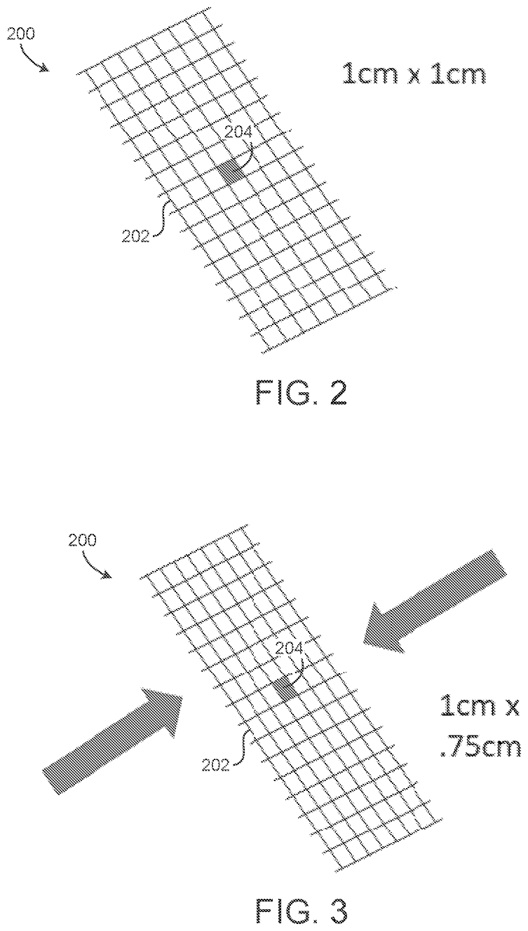

Referring now to FIGS. 2-3, 4A-4C, and 5A-5C, various pattern indicators are shown, according various exemplary embodiments. It should be understood that any of the pattern indicators described herein with reference to FIGS. 2-3, 4A-4C, and 5A-5C may be the same as or similar to the pattern indicators 118. FIGS. 2-3 show a pattern indicator 200 that includes a reference indicator 204 and a grid 202, according to some embodiments. The reference indicator 204 may be a rigid member, a colored member, etc., that can be used in an image processing technique to identify orientation of an image capture device relative to the pattern indicator 200. The reference indicator 204 may be configured to maintain shape and size (e.g., the reference indicator 204 does not deform) as the grid 202 deforms (e.g., increases or decreases in size, decreases in width, etc.). Advantageously, the reference indicator 204 can facilitate normalizing different image data so that image data of the pattern indicator 200 can be compared to each other, regardless of a relative orientation or distance between the imaging device and the pattern indicator 200 at which the image data is obtained.

As shown in FIGS. 2-3, the pattern indicator 200 deformed in a lateral direction (e.g., width-wise), according to some embodiments. The pattern indicator 200 may deform or decrease in width or in the lateral direction due to swelling reduction of a portion of the patient's limb at which the pattern indicator 200 is positioned. Specifically, each cell of the grid 202 may initially have dimensions of 1 cm by 1 cm (shown in FIG. 2). At some point later (e.g., when an image of the pattern indicator 200 is obtained), the grid 202 may deform (e.g., decrease in width or decrease in overall size in a lateral direction) as shown in FIG. 3, so that each cell of the grid 202 has dimensions of 1 cm by 0.75 cm. This change in width of the cells of the grid 202 may occur due to decreased swelling of the portion of the patient's limb where the pattern indicator 200 is positioned.

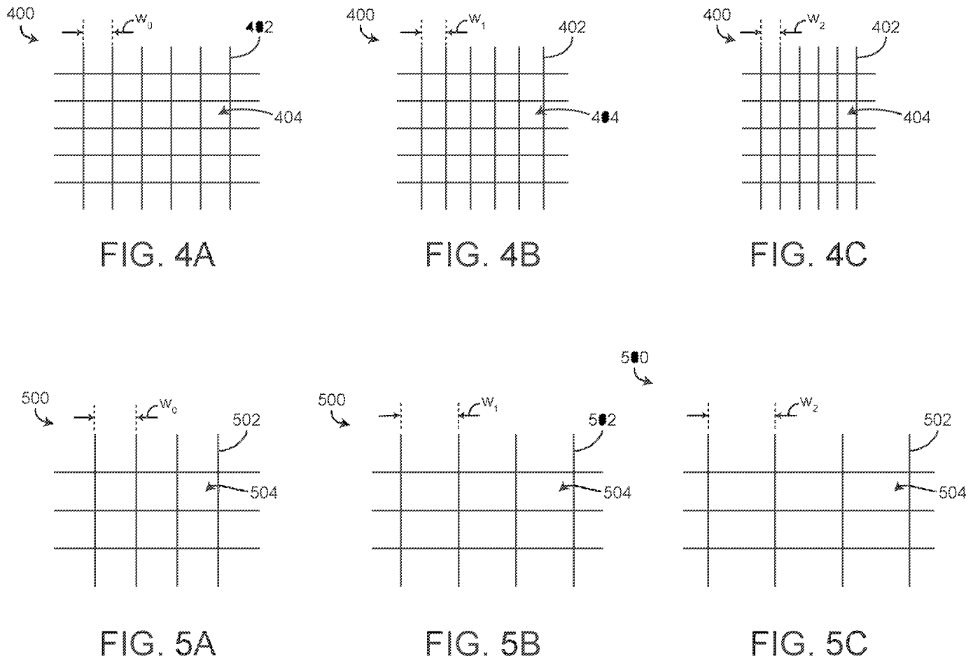

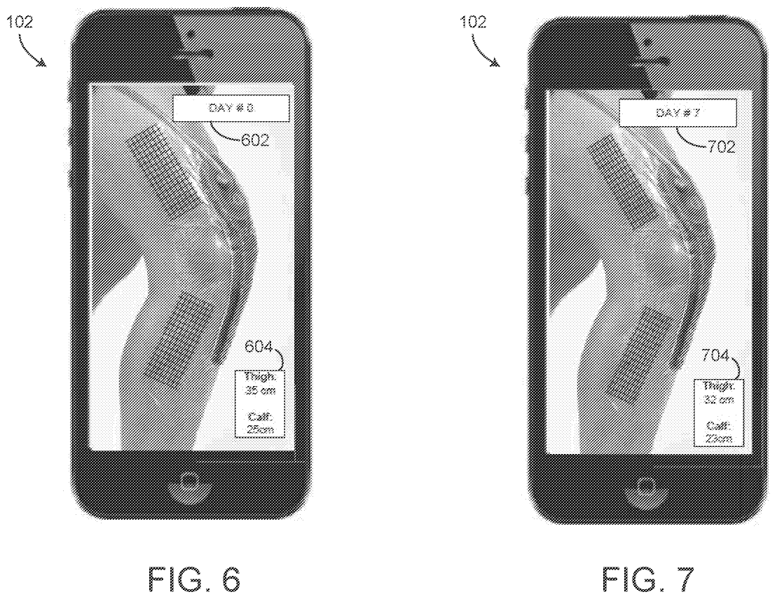

Referring particularly to FIGS. 4A-4C, another pattern indicator 400 is shown, according to some embodiments. FIGS. 4A-4C illustrate change in the pattern indicator 400 when swelling of the limb at which the pattern indicator 400 is positioned decreases over time. The pattern indicator 400 may include a grid 402 having multiple cells 404 defined by lines of the grid 402. Horizontal lines of the grid 402 may define a lateral or width-wise direction, while vertical lines of the grid 402 may define a longitudinal, vertical, or height-wise direction. The cells 404 of the grid 402 may initially have a width w₀. After healing of a wound site at which the pattern indicator 400 is positioned progresses, swelling may reduce, thereby causing a decrease in diameter and/or circumference of the patient's limb where the pattern indicator 400 is positioned. In some embodiments, the horizontal lines of the grid 402 are aligned with a circumferential direction of the patient's limb so that decreases in the circumference of the patient's limb cause decreases in the width of the cells 404. In other applications, the horizontal lines of the grid 402 are angularly offset from the circumferential direction so that decreases in the circumference of the patient's limb cause changes in the width and a height of the cells 404, or cause the cells 404 to skew. FIG. 4A may represent a baseline or an initial dimension of the cells 404, w₀ (e.g., before NPWT is provided to a wound).

Referring now to FIGS. 4B and 4C, the pattern indicator 400 is shown at two different later times when swelling reduction has occurred or when a change to the circumference or diameter of the patient's limb has occurred, according to some embodiments. The width of the cells 404 of the pattern indicator 400 is shown decreased from w₀ to w₁ between FIGS. 4A and 4B, and from w₁ to w₂ between FIGS. 4B and 4C (or from w₀ to w₂ between FIGS. 4A and 4C). An image of the pattern indicator 400 as shown in FIG. 4A may be obtained at time t₀ as a baseline or reference image. An image of the pattern indicator 400 as shown in FIG. 4B may be obtained at time t₁, and another image of the pattern indicator 400 as shown in FIG. 4C can be obtained at time t₂. An image processing technique may be performed to determine values of the widths w₀, w₁, and w₂ at the times t₀, t₁, and t₂ and relative changes in the widths therebetween.

Referring now to FIGS. 5A-5C, another pattern indicator 500 is shown, according to some embodiments. FIGS. 5A-5C illustrate changes in the pattern indicator 500 when swelling of the limb at which the pattern indicator 500 is positioned increases over time. The pattern indicator 500 may include a grid 502 having multiple cells 504 defined by lines of the grid 502. Horizontal lines of the grid 502 may define a lateral or width-wise direction, while vertical lines of the grid 502 may define a longitudinal, vertical, or height-wise direction. The cells 504 of the grid 502 may initially have a width w₀ as shown in FIG. 5A. Over time, swelling may increase at a wound site at which the pattern indicator 500 is positioned, thereby causing an increase in diameter and/or circumference of the patient's limb where the pattern indicator 500 is positioned. In some embodiments, the horizontal lines of the grid 502 are aligned with a circumferential direction of the patient's limb so that increase in the circumference of the patient's limb cause increases in the width of the cells 504. In other applications, the horizontal lines of the grid 502 are angularly offset relative to the circumferential direction so that increases in the circumference of the patient's limb cause change in the width and a height of the cells 504, or cause the cells 504 to skew. FIG. 5A may represent a baseline or initial dimension of the cells 504, w₀.

Referring now to FIGS. 5B and 5C, the pattern indicator 500 is shown at two different later times when swelling has increased, or when a change in the circumference or diameter of the patient's limb has occurred, according to some embodiments. The width of the cells 504 of the pattern indicator 500 are shown increased from w₀ to w₁ between FIGS. 5A and 5B, and from w₁ to w₂ between FIGS. 5B and 5C (or from w₀ to w₂ between FIGS. 5A and 5C). An image of the pattern indicator 500 as shown in FIG. 5A may be obtained at time to, and another image of the pattern indicator 500 as shown in FIG. 5C can be obtained at time t₂. An image processing technique may be performed to determine values of the widths w₀, w₁, and w₂ at the times t₀, t₁, and t₂, and relative changes in the widths therebetween.

Referring generally to FIGS. 1-5C, any of the pattern indicators 118, 200, 400, or 500 may be provided as a printed grid (e.g., a flexible grid) that is pre-placed on the dressing 110 at a desired location. The pattern indicators 118, 200, 400, or 500 may be printed onto the dressing 110 (e.g., onto an outer drape or later of the dressing 110) so that the pattern indicators 118, 200, 400, or 500 are viewable by the imaging device 106. The pattern indicators 118, 200, 400, or 500 may have a fixed square area that is flexible so that overall geometry, shape, size, width, height, skew, etc., of the grids thereof changes with changes to the patient's limb 160. In other embodiments, the pattern indicators 118, 200, 400, or 500 are provided to a caregiver as a part of a kit and the caregiver or clinician may provide and place the pattern indicator 118, 200, 400, or 500 in a desired location (e.g., near a patient's wound). The pattern indicator 118, 200, 400, or 500 may be placed or adhered to an exterior surface of the dressing 110, or may be adhered directly to the patient's skin if the wound drape 114 is clear or transparent. In some embodiments, the pattern indicator 118, 200, 400, or 500 is flexible but does not stretch laterally or longitudinally. Such a configuration or material may ensure that grid spacing is preserved during application of the pattern indicator 118, 200, 400, or 500. In some embodiments, the pattern indicator 118, 200, 400, or 500 is provided with a grid stabilization layer. Once the pattern indicator 118, 200, 400, or 500 is placed at a desired location, the grid stabilization layer can be removed so that the pattern indicator 118, 200, 400, or 500 can expand or contract with the patient's skin. The pattern indicator 118, 200, 400, or 500 can be preferably positioned on or near a flat surface of the patient's limb 160. In some embodiments, if the pattern indicator 118, 200, 400, or 500 is positioned on a curved portion of the patient's limb 160, curvature of the pattern indicator 118, 200, 400, or 500 can be accounted for by the smartphone 102 using any of the techniques described herein.

### Other Pattern Indicators

Referring generally to the FIGURES, it should be understood that while the systems and methods herein are described as including a grid pattern, any of the systems, methods, dressings, etc., herein may have different patterns other than a grid. For example, the pattern indicator 118 may be a sticker, a grid, a predetermined shape (e.g., a square, a circle, etc.), a set of lines, etc. In another example, the pattern indicator can be a white square with a black shape to provide contrast therebetween, which can be monitored and tracked to determine healing or swelling changes. Similarly, while the reference indicator 204 is described herein as a rigid red square, the reference indicator 204 may have other shapes (e.g., a polygon, a circle, a star, etc.), or other colors or materials (e.g., a neon color, a reflective material, a limb green or bright yellow color, etc.). Additionally, while the pattern indicators described herein are described as being a component of the dressing, the pattern indicators may be additional components that are placed and adhered to the dressing or the patient's skin.

### System Diagram

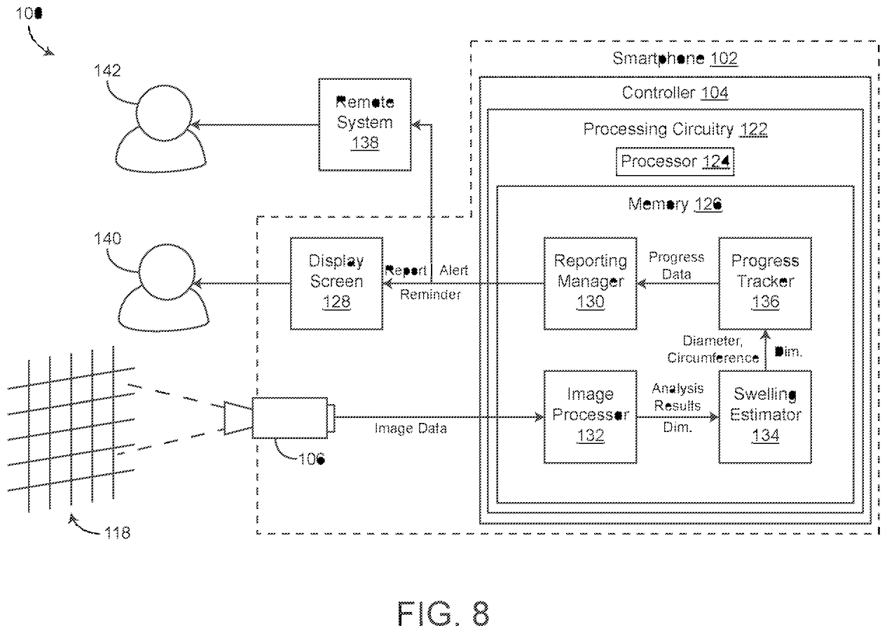

Referring now to FIG. 8, the system 100 is shown in greater detail, according to some embodiments. Specifically, FIG. 8 shows the controller 104 of the smartphone 102 and the functionality thereof in greater detail. The controller 104 is shown to include processing circuitry 122 including a processor 124 and memory 126. The processor 124 may be a general purpose single- or multi-chip processor, a digital signal processor (DSP), an application specific integrated circuit (ASIC), a field programmable gate array (FPGA), or other programmable logic device, discrete gate or transistor logic, discrete hardware components, or any combination thereof designed to perform the functions described herein. The processor 124 may be a microprocessor, or, any conventional processor, controller, microcontroller, or state machine. The processor 124 also may be implemented as a combination of computing devices, such as a combination of a DSP and a microprocessor, a plurality of microprocessors, one or more microprocessors in conjunction with a DSP core, or any other such configuration. In some embodiments, particular processes and methods may be performed by circuitry that is specific to a given function.

The memory 126 (e.g., memory, memory unit, storage device) may include one or more devices (e.g., RAM, ROM, Flash memory, hard disk storage) for storing data and/or computer code for completing or facilitating the various processes, layers and modules described in the present disclosure. The memory 126 may be or include volatile memory or non-volatile memory, and may include database components, object code components, script components, or any other type of information structure for supporting the various activities and information structures described herein. According to an exemplary embodiment, the memory 126 is communicably connected to the processor 124 via the processing circuitry 122 and includes computer code for executing (e.g., by the processing circuit or the processor) the one or more processes described herein.

The controller 104 of the smartphone 102 is configured to communicate with the imaging device 106 of the smartphone 102, a display screen 128 of the smartphone 102, or a remote system 138, according to some embodiments. The memory 126 includes an image processor 132, a swelling estimator 134, a progress tracker 136, and a reporting manager 130. The image processor 132 is configured to obtain image data from the imaging device 106 and perform an image analysis technique to determine a dimension of the pattern indicator 118. The swelling estimator 134 is configured to use analysis results of the image processor 132 and/or the dimension of the pattern indicator 118 to determine different swelling estimations or to determine different properties or dimensions of a patient's limb (e.g., a diameter, a circumference, etc.). The progress tracker 136 is configured to obtain and store the dimension from the swelling estimator 134 and generate progress data, according to some embodiments. The progress data can be provided to the reporting manager 130 where it is used to generate a report, an alert, or a reminder.

The image processor 132 may be configured to obtain image data from the imaging device 106 and perform an image analysis or processing technique. The image data may be generated when a patient directs the smartphone 102 towards the pattern indicator 118 (e.g., at an approximately orthogonal orientation) and captures an image. The image processor 132 can be configured to use the image data and generate a point cloud at intersections of lines (e.g., grid lines) of the pattern indicator 118. The image processor 132 may use the reference indicator 204 (e.g., a rigid reference square, one or more rigid reference squares or points, etc.) to compensate for or account for relative orientation and/or position or distance between the imaging device 106 and the pattern indicator 118. In some embodiments, the image processor 132 is configured to determine a relative orientation and/or position between the imaging device 106 and the pattern indicator 118 for baseline image data. The image processor 132 can use the relative orientation and/or position between the imaging device 106 and the pattern indicator 118 of the baseline image data as a reference relative orientation and/or position. Subsequently obtained image data can be skewed, normalized, etc., or otherwise calibrated using newly determined relative orientation and/or position between the imaging device 106 and the pattern indicator 118 (e.g., by determining relative position of corners of the reference indicator 204).

For example, when the image processor 132 obtains baseline image data (e.g., of a baseline image), the image processor 132 can obtain a baseline relative orientation θ_{ref} and a baseline relative position p_{ref} between the imaging device 106 and the pattern indicator 118. Subsequently or newly captured image data may be processed by the image processor 132 to determine relative orientation θ_{new} and relative position p_{new} between the imaging device 106 and the pattern indicator 118. If the relative orientation θ_{new} and the relative position p_{new} of the newly captured image data does not match the baseline orientation θ_{ref} and the baseline relative position p_{ref}, the image processor 132 can be configured to calibrate, modify, adjust, etc., the newly captured image data so that the relative orientation θ_{new} and the relative position p_{new} are equal to or the same as the baseline relative orientation θ_{ref} and the baseline relative position p_{ref}. The calibrated image data can then be used by the image processor 132 to obtain analysis results and/or a dimension of the pattern indicator 118.

In some embodiments, the image processor 132 is configured to calibrate the baseline image data and all newly captured image data to have an orthogonal relative orientation θ_{⊥} and predetermined distance p_{d}. For example, if the image processor 132 obtains image data and determines (using the detected position and/or skew of the reference indicator 204) that the relative orientation θ and the relative position or distance p of the newly captured image data does not match the orthogonal relative orientation θ_{⊥} and predetermined distance p_{d}, the image processor 132 can modify, adjust, calibrate, etc., the image data so that the relative orientation θ of the image data and the relative position p of the image data matches or is equal to the orthogonal relative orientation θ_{⊥} and the predetermined distance p_{d}. The image processor 132 can then use the calibrated image data to generate the analysis results and/or the dimension.

In some embodiments, the image processor 132 is configured to analyze the image data (calibrated image data, baseline image data, newly captured image data, etc.) and determine a dimension dim of the pattern indicator 118. In some embodiments, the image processor 132 does not adjust or calibrate the image data directly, but uses the relative position p and/or the relative orientation θ_{⊥} to adjust or determine a calibration for the dimension.

The dimension dim can be a width of one or more of the cells of the pattern indicator 118, a height of one or more of the cells of the pattern indicator 118, a size of one or more of the cells of the pattern indicator 118, an average width of one or more cells of the pattern indicator 118, an average height of one or more of the cells of the pattern indicator 118, an average size of one or more of the cells of the pattern indicator 118, etc. In some embodiments, the dimension dim is provided to the swelling estimator 134 for use in determining swelling changes, or other dimension changes of the patient's limb (e.g., diameter, circumference, etc.).

The image processor 132 can be configured to use a point cloud generation technique to generate multiple points at intersections of different lines of the pattern indicator 118. The image processor 132 can also generate points for the reference indicator 204 (e.g., four vertices or points for the four corners of the reference indicator 204 if the reference indicator 204 is a square). The image processor 132 can therefore obtain relative orientation and/or relative position of the pattern indicator 118 relative to the imaging device 106. The image processor 132 can use the generated point cloud to determine the dimension dim of the pattern indicator 118. The image processor 132 can output analysis results from the point cloud generation technique and/or the dimension dim to the swelling estimator 134.

The swelling estimator 134 is configured to use the dimension dim and/or the analysis results to determine or more swelling properties, or one or more properties of the patient's limb at which the pattern indicator 118 is located, according to some embodiments. For example, the swelling estimator 134 can be configured to use the dimension dim of the pattern indicator 118 to estimate a diameter D of the patient's limb, a circumference C of the patient's limb, a volume V of the patient's limb, etc. In some embodiments, the swelling estimator 134 uses a known size of the pattern indicator 118, or known values of the dimension dim of the pattern indicator 118 to estimate the diameter D, circumference C, volume V, etc.

The swelling estimator 134 can also be configured to use changes in the dimension Δdim to determine changes in the diameter ΔD, the circumference ΔC or changes in the volume ΔV. For example, if the dimension dim changes by a specific amount, the swelling estimator 134 can be configured to use a function, a set of equations, a relationship, a graph, etc., and known properties or dimensions of the patient's limb or the pattern indicator 118 in combination with the dimension dim or the change in the dimension Δdim (between subsequently captured image data, or between baseline image data and currently captured image data, etc.) to determine the changes in the diameter ΔD, changes in the circumference ΔC, or changes in the volume ΔV of the patient's limb.

The swelling estimator 134 can store initial known values of any of a verity of dimensions of the patient's limb. For example, the swelling estimator 134 can store known initial values of the circumference, the volume, the diameter, etc., of the patient's limb. These values can be obtained by performing a manual measurement technique of the patient's limb to obtain values of the circumference, the volume, the diameter, etc., of the patient's limb. These values may be provided to the smartphone 102 via the remote system 138 or through the display screen 128.

The progress tracker 136 is configured to obtain any of the dimension dim, the change in the dimension Δdim (e.g., between subsequently captured image data, between baseline image data and currently captured image data, etc.), a currently estimated diameter D of the patient's limb, a currently estimated circumference C of the patient's limb, a currently estimated volume V of the patient's limb, a change in any of the diameter ΔD, the circumference ΔC, the volume ΔV, etc., of the patient's limb. In some embodiments, the progress tracker 136 is configured to estimate changes in different dimensions or measurements of the patient's limb. For example, the progress tracker 136 may obtain currently estimated values of the diameter D, the circumference C, or the volume V of the patient's limb and estimate changes (e.g., between the currently estimated values of the diameter D, the circumference C, or the volume V and previously estimated values of the diameter D, the circumference C, or the volume V or between the currently estimated values of the diameter D, the circumference C, or the volume V and the initial or baseline values of the diameter D, the circumference C, or the volume V) in any of the diameter D, the circumference C, or the volume V of the patient's limb. In some embodiments, the progress tracker 136 is configured to estimate or calculate percentage changes in any of the circumference, the diameter, the volume, the dimension, etc., of the patient's limb relative to baseline or reference or initial values.

The progress tracker 136 can also obtain and store values of the dimension dim over time. The progress tracker 136 can store any of the values described herein as time-series data with a corresponding time and/or date at which the values are obtained. In some embodiments, the progress tracker 136 is configured to provide any of the stored data to the reporting manager 130 as progress data.

The reporting manager 130 is configured to use the progress data provided by the progress tracker 136 to generate a report, an alert, or a reminder, according to some embodiments. The reporting manager 130 can provide the report to a display screen 128 so that the display screen 128 of the smartphone 102 operates to provide the report to a patient 140. In some embodiments, the reporting manager 130 is configured to provide the report to a remote system 138 so that the remote system 138 can provide the report to a healthcare professional 142. In this way, the healthcare professional 142 can monitor and track swelling or healing progress of the patient 140. The reporting manager 130 may communicate with the remote system 138 via a cellular network, the Internet, etc. The reporting manager 130 can provide the report, the alert, or the reminder to the remote system 138 or the display screen 128 in response to a request, at periodic time intervals, in response to the progress data indicating increased swelling, etc.

The smartphone 102 can be used by the patient 140 to obtain periodic image data of the pattern indicator 118. For example, the reporting manager 130 can check the progress data to determine if new image data should be obtained. If the progress data indicates that new image data should be obtained (e.g., every day, every other day, twice a day, etc.), the reporting manager 130 can operate the display screen 128 to provide a reminder to the patient 140 to capture image data of the pattern indicator 118. Image data, analysis results, dimensions of the pattern indicator 118, swelling parameters or dimensions (e.g., diameter, circumference, volume, etc.) can be obtained at scheduled times of day, and may be stored in the progress tracker 136 with associated date and time.

The reporting manager 130 and/or the progress tracker 136 can also analyze the progress data to identify any alert conditions. For example, the alert conditions may be any conditions that indicate an increase in swelling (e.g., a significant increase in diameter, circumference, volume, etc., of the patient's limb). In response to determining that the progress data indicates an increase in swelling, the reporting manager 130 can provide the alert any of the patient 140 (e.g., by operating the display screen 128 to provide a visual and/or an aural alert), or the healthcare professional 142 (e.g., by providing the alert to the remote system 138).

The patient 140 can use the smartphone 102 to periodically capture the image data so that the progress tracker 136 can compile the progress data. The progress data may indicate swelling trends, healing progression, changes in the different dimensions of the patient's limb, etc.

### Mobile Application

Referring still to FIG. 8, the smartphone 102 can be configured to perform the functionality described herein when the smartphone 102 is loaded with a mobile application. The mobile application can be downloaded onto the smartphone 102 (e.g., an APK file, an application file, etc.) and installed on the smartphone 102 to configure the smartphone 102 or the processing circuitry 122 of the smartphone 102 to perform any of the techniques described herein. In some embodiments, the mobile application requires the patient 140 to provide access to the imaging device 106 of the smartphone 102, communicative abilities of the smartphone 102 (e.g., so that the smartphone 102 can communicate with the remote system 138), access to alerting features of the smartphone 102 (e.g., so that the reporting manager 130 can provide alerts, notifications, reminders, etc., to the patient 140 via the display screen 128).

### Process

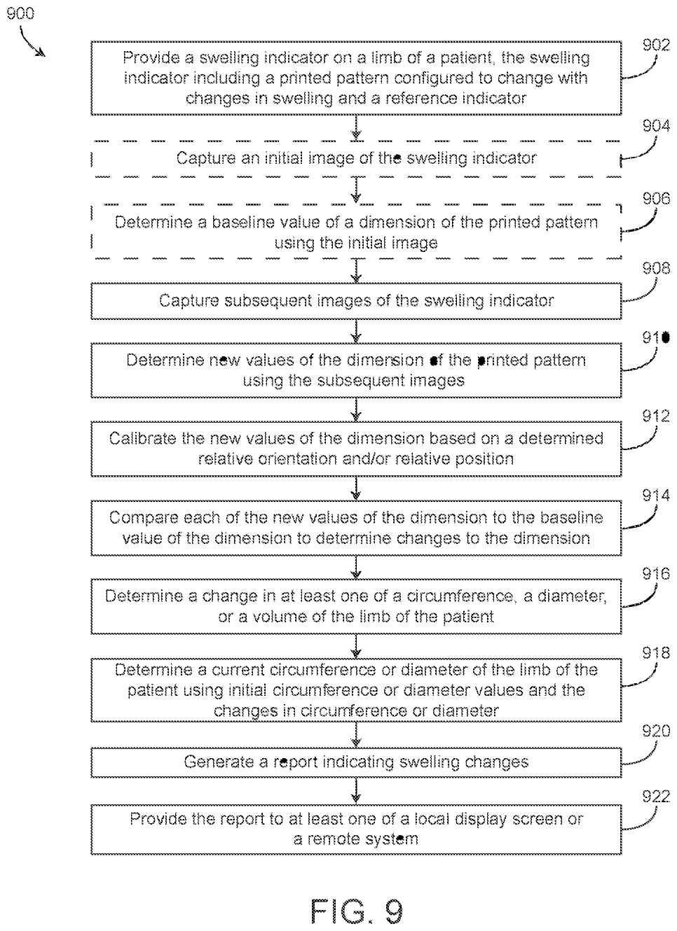

Referring now to FIG. 9, a flow diagram of a process 900 for monitoring and tracking healing progression or swelling reduction of a patient's limb is shown, according to some embodiments. Process 900 includes steps 902-920 and can be performed by system 100. In some embodiments, process 900 is performed at least partially by a patient to obtain image data of a pattern indicator (e.g., a grid) so that swelling reduction can be quantified and tracked.

Process 900 includes providing a swelling indicator on a limb of a patient, the swelling indicator including a printed pattern configured to change with changes in swelling and a reference indicator (step 902), according to some embodiments. Step 902 can be performed by a caregiver by providing a wound dressing (e.g., the dressing 110) at a wound site (e.g., at a knee of a patient's limb). The printed pattern can be a grid with intersecting lines. The reference indicator can be a rigid member that does not deform or change as the printed pattern stretches or changes due to swelling reduction or increase. The swelling indicator can be printed onto a layer of the wound dressing so that captured images of the patient's limb include the swelling indicator. The swelling indicator may be positioned proximate a portion of the patient's limb where swelling tracking is desired.

Process 900 includes capturing an initial image of the swelling indicator (step 904), according to some embodiments. Step 904 can be performed by the patient using a smartphone, an imaging device, a personal computer device, etc. For example, the patient may direct or point the smartphone towards the swelling indicator and operate the smartphone to capture the initial image of the swelling indicator. In other embodiments, step 904 is performed by a caregiver to capture the initial image. The initial image may be captured with the smartphone positioned a predetermined distance (e.g., 12 inches, etc.) from the swelling indicator and at a substantially orthogonal or perpendicular orientation relative to the patient's limb.

Process 900 includes determining a baseline value of a dimension of the printed pattern using the initial image (step 906), according to some embodiments. Step 906 can be performed by processing circuitry of the smartphone, or more particularly by the image processor 132. Step 906 can include generating a point cloud at intersections of lines of the printed pattern. The point cloud can be part of an image analysis technique. Step 906 can be performed by determining a width or a height of one or more cells of the printed pattern (e.g., the width w₀). In some embodiments, steps 904 and 906 are optional. For example, if the reference indicator is a known dimension (such as a 1 cm×1 cm red square), and the printed pattern of the swelling indicator is a grid with known dimensions in a recently pre-placed location, capturing the initial image and determining the baseline value using the initial image may be optional.

Process 900 includes capturing subsequent images of the swelling indicator (step 908) and determining new values of the dimension of the printed pattern using the subsequent images (step 910), according to some embodiments. Steps 908 and 910 can be performed similarly to steps 904 and 906. For example, the subsequent images of the swelling indicator (step 908) and the new values of the dimensions of the printed pattern (step 910) can be obtained or the steps performed at a time after the steps 904 and 906 are performed (e.g., a day later, a time interval later, etc.).

Process 900 includes calibrating the new values of the dimension based on a determined relative orientation and/or relative position (step 912), according to some embodiments. The relative orientation and/or the relative position may be a relative orientation and/or position of an imaging device that is used to capture the images of the swelling indicator, and the swelling indicator or limb of the patient. The new values of the dimension can be calibrated to account for differences in the relative orientation and/or position of the imaging device relative to the patient's limb for different images. In some embodiments, step 912 is performed to calibrate different calculated swelling values (e.g., circumference, diameter, volume, etc.) of the patient's limb. Step 912 can be performed by the smartphone, the processing circuitry thereof (e.g., smartphone 102 and/or processing circuitry 122) or, more particularly, by the image processor 132 using any of the techniques described in greater detail above with reference to FIG. 8.

Process 900 includes comparing each of the new values of the dimension to the baseline value of the dimension to determine changes to the dimension (step 914), according to some embodiments. Step 914 can be performed by the smartphone (e.g., the smartphone 102), processing circuitry of the smartphone, or more particularly by the swelling estimator 134 or the progress tracker 136. Step 914 can include determining a difference between the new values of the dimension and the baseline value, or determining percent changes (e.g., total percent increase or total percent decrease) of the new values relative to the baseline values.

Process 900 includes determining a change in at least one of a circumference, a diameter, or a volume of the limb of the patient (step 916), according to some embodiments. Step 916 can be performed by the swelling estimator 134 and/or the progress tracker 136. Step 916 may include determining an increase or decrease amount of any of the circumference, the diameter, or the volume of the limb of the patient, a current value of any of the circumference, the diameter, or the volume of the limb of the patient, a percent increase (e.g., relative to a baseline or initial value or relative to a previous value) of any of the circumference, the diameter, or the volume of the limb of the patient, etc. The change in any of the circumference, the diameter, or the volume of the limb can be estimated using changes in the dimension of the swelling indicator. For example, as swelling at the patient's limb reduces, a width of the swelling indicator may also decrease (e.g., if a width-wise direction of the swelling indicator is parallel with or aligned with a circumferential direction of the patient's limb). This decrease in width of the swelling indicator can thereby be used to estimate or calculate a decrease in the circumference, a decrease in the diameter, a decrease in the volume, etc., of the patient's limb.

Process 900 includes determining a current circumference or diameter of the limb of the patient using initial circumference or diameter values and the changes in circumference or diameter (step 918), according to some embodiments. Step 918 can also include determining a current volume of the limb of the patient. Step 919 may include using the changes in any of the circumference, diameter, or volume of the limb and the initial circumference, diameter, or volume of the limb to determine the current circumference diameter, or volume of the limb (e.g., by adding or subtracting the changes to the initial values) or may include directly estimating any of the current circumference, diameter, or volume of the patient's limb using currently captured image data. The reference indicator may be a known size (e.g., a 1 cm by 1 cm square) and can be used to determine actual size or dimension values of the patient's limb as opposed to relative values (e.g., relative decreases or increases between subsequently captured images, or between a currently captured image and an initial or baseline image).

Process 900 includes generating a report indicating swelling changes (step 920), according to some embodiments. The report can include graphs, time-series data, total percent swelling reduction (e.g., quantified in any of circumference, diameter, volume, etc., of the patient's limb), relative swelling reduction (e.g., percent reduction or absolute reduction between subsequent time-series data points of any of the circumference, diameter, volume, dimension of the pattern, etc.), patient information, alert statuses, therapy information, etc. Step 920 can be performed based on progress data that is obtained and stored over time as additional and subsequent images of the printed pattern are obtained, and as new values of the dimension of the printed pattern, or any of the circumference, the diameter, or the volume (or changes therein) of the limb of the patient are obtained. Step 920 can be performed by the reporting manager 130 of the smartphone 102.

Process 900 includes providing the report to at least one of a local display screen or a remote system (step 922), according to some embodiments. The local display screen may be the display screen 128 (e.g., a user interface or a display screen of the smartphone). The remote system may be the remote system 138 (e.g., a remote caregiver system, a hospital system, a remote server, a webpage, etc.) where a healthcare or medical professional or a caregiver can view the report.

### Progress Graphs

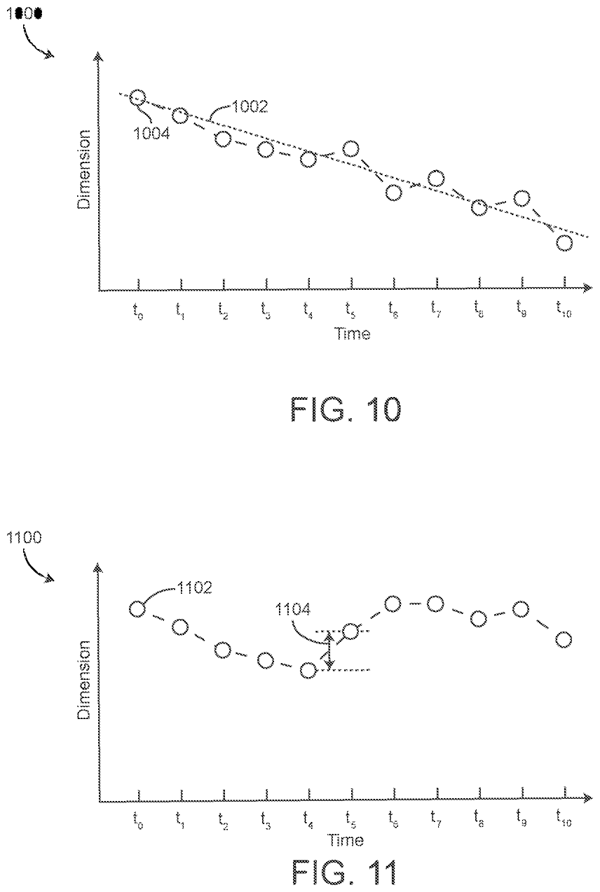

Referring now to FIGS. 10 and 11, different progress graphs 1000 and 1100 are shown, according to some embodiments. The progress graph 1000 shows progression of a dimension over time (e.g., a width of a pattern indicator or graph that is coupled on a patient's limb, a volume of the patient's limb, a dimension or measurement of a pattern indicator or graph that is coupled on a patient's limb, a circumference of the patient's limb, a diameter of the patient's limb, etc.) as swelling reduction of the patient's limb occurs. The progress graph 1100 shows progression of a dimension over time (e.g., a width of a pattern indicator or graph that is coupled on a patient's limb, a volume of the patient's limb, a dimension or measurement of a pattern indicator or graph that is coupled on a patient's limb, a circumference of the patient's limb, a diameter of the patient's limb, etc.) as swelling of the patient's limb increases at time t=t₄.

Referring particularly to FIG. 10, the progress graph 1000 includes a Y-axis illustrating a dimension (e.g., a width of a cell of a pattern indicator, a circumference, diameter, or a volume) and an X-axis illustrating time or date progression including different time steps (e.g., t₀, t₁, t₂, . . . , t₁₀). As shown in FIG. 10, the progress graph 1000 includes scatter data 1004 including different scatter points or scatter data. Each of the scatter points is associated with a different time step and illustrates a value of the dimension at the time at which the value of the dimension is obtained. As shown in FIG. 10, the scatter data 1004 illustrates a downward trend in the dimension over time, thereby indicating decreased swelling, or decreased size of the patient's limb over time. The progress graph 1000 can also include a trendline 1002 that is generated to demonstrate a trend of the scatter data 1004 over time. The trendline 1002 may be a linear trendline and an equation of the linear trendline (e.g., a slope) can be used to estimate when the dimension will reach a desired amount, or when swelling will have reduced by a desired amount. The progress graph 1000 can be generated by the reporting manager 130 as part of the report and may be provided to the display screen 128 and/or the remote system 138.

Referring particularly to FIG. 11, the progress graph 1100 also includes a Y-axis illustrating a dimension (e.g., a width of a cell of a pattern indicator, a circumference, diameter, or a volume) and an X-axis illustrating time or date progression including different time steps (e.g., t₀, t₁, t₂, . . . , t₁₀). As shown in FIG. 11, the progress graph 1100 includes scatter data 1102 including different scatter points or scatter data. Each of the scatter points is associated with a different time step and illustrates a value of the dimension at the time at which the value of the dimension is obtained. As shown in FIG. 11, the scatter data 1102 illustrates a downward trend up to time t=t₄, after which the scatter data 1102 illustrates an increase in the dimension. Specifically, the dimension is shown increasing by an amount 1104 from time t=t₄ to time t=t₅. If the amount 1104 exceeds a predetermined amount, or if a difference between the dimension at time t=t₄ and the time t=t₆ still exceeds the predetermined amount, the reporting manager 130 may determine that the swelling at the patient's limb has increased by an undesired amount and can provide an alert to the remote system 138 and/or the display screen 128.

In some embodiments, the predetermined amount is an increase or a decrease amount that is set by a clinician or a healthcare professional. If the dimension (e.g., the circumference, the diameter, the volume, a dimension of the pattern indicator or grid, etc.) increases by more than the predetermined increase amount, this may indicate an undesired increase in swelling and the patient can be alerted (e.g., via the display screen 128 or via an aural alert of the smartphone 102) and a healthcare professional may also be alerted (e.g., via the remote system 138). If the dimension (e.g., the circumference, the diameter, the volume, a dimension of the pattern indicator or grid, etc.) decreases by more than the predetermined decrease amount, this may indicate that a substantial swelling reduction has occurred and the healthcare professional may be notified via the remote system 138 and/or the patient can be notified via the display screen 128. It should be understood that while the amount 1104 is shown as a decrease or an increase between consecutive time-steps or measurements, the amount 1104 may also be an increase or a decrease amount of a current data point (e.g., a current value of the circumference of the patient's limb) relative to a baseline data point (e.g., an initial or baseline value of the circumference of the patient's limb).

### Pre-Operative Application

Referring particularly to FIG. 10 and FIG. 8, the progress tracker 136 and/or the reporting manager 130 can be configured to perform a regression technique using the progress data to determine a trend of the progress data (e.g., to determine an equation of the trendline 1002). In some embodiments, the progress tracker 136 and/or the reporting manager 130 are configured to use the trend or an equation thereof to predict when a dimension of the patient's limb will reach a target amount. The target amount may indicate a time at which swelling has reduced to a desired amount. The target amount can be set by a healthcare professional. For example, if a patient breaks his/her ankle and requires surgery, but a degree of swelling at the patient's ankle does not allow the surgery, the progress tracker 136 and/or the reporting manager 130 can be configured to predict a time at which the swelling will be sufficiently low to perform the surgery. The reporting manager 130 can provide a predicted time t_{predict} at which the dimension will reach a target value to the remote system 138 so that the remote system can pre-emptively schedule the surgery for the patient. In some embodiments, the reporting manager 130 provides the progress data and/or the equation of the trend of the progress data to the remote system 138 for use in pre-emptive scheduling.

### Example Report

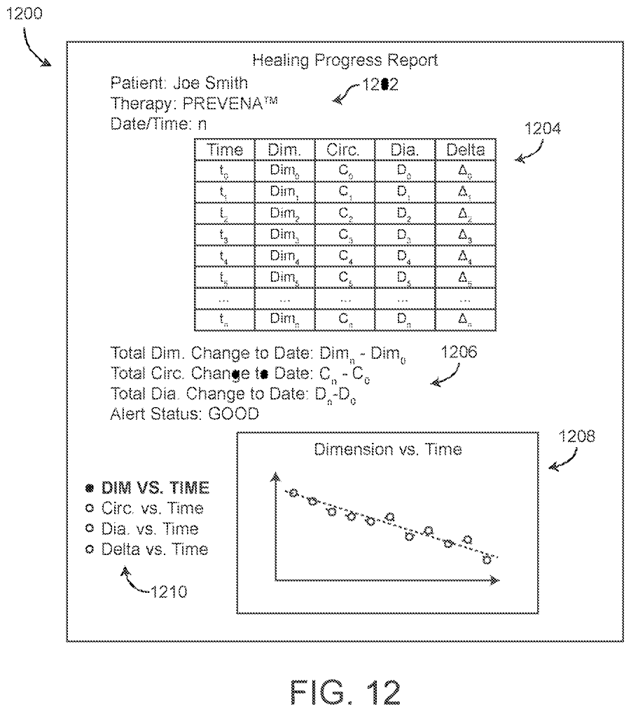

Referring now to FIG. 12, a progress report 1200 is shown, according to some embodiments. The progress report 1200 as shown in FIG. 12 may be shown to the patient 140 via the display screen 128 and/or may be provided to the healthcare professional 142 via the remote system 138. The progress report 1200 includes patient information 1202 (e.g., patient name, patient date of birth, patient age, NPWT location or application, therapy type, date and time, etc.), healing progress data (e.g., in a tabular form) 1204 which may include time of recorded data, dimension of the pattern indicator, circumference of the patient's limb, diameter of the patient's limb, volume of the patient's limb, a percent or absolute increase or decrease of any of the dimension, circumference, diameter, or volume, etc., current status information 1206 (e.g., total percent or absolute dimension change to date, total percent or absolute circumference change to date, total percent or absolute diameter change to date, total percent or absolute volume change to date, current alert status, etc.), a set of menus 1210, and/or a graph 1208.

The graph 1208 may be any of graph 1000 or graph 1100. In some embodiments, the graph 1208 is a graphical representation illustrating different changes to the patient's limb or swelling changes (e.g., increases or decreases) over time. For example, the graph 1208 may be a graphical representation of any of the dimension, the circumference, the diameter, or the volume over time. The menus 1210 may be user-selectable and may show different dimensions or properties of the patient's limb over time. For example, the menus 1210 can include different selectable options of the dimension of the pattern indicator over time, the circumference of the patient's limb over time, the diameter of the patient's limb over time, the volume of the patient's limb over time, or a change of any of the dimension, the diameter, the circumference, or the volume over time. The progress report 1200 may also include any captured images that are obtained by the imaging device 106 in order to generate, estimate, calculate, etc., the healing progress data 1204.

### Advantages

Advantageously, the systems and methods described herein can be used to automatically track and monitor healing progression of swelling reduction of a patient's limb. The systems and methods can be implemented by use of a patient's smartphone loaded with a mobile application. This provides an automatic and improved monitoring system, and facilitates ease of measurement of swelling of the patient's limb. Other techniques require manual measurement, dunking of the patient's limb, etc., to determine swelling changes. The systems and methods described herein are advantageous because they provide a more automatic approach to quantifying, tracking, and monitoring swelling of a patient's limb over time.

### Configuration of Exemplary Embodiments

As utilized herein, the terms “approximately”, “about”, “substantially”, and similar terms are intended to have a broad meaning in harmony with the common and accepted usage by those of ordinary skill in the art to which the subject matter of this disclosure pertains. It should be understood by those of skill in the art who review this disclosure that these terms are intended to allow a description of certain features described and claimed without restricting the scope of these features to the precise numerical ranges provided. Accordingly, these terms should be interpreted as indicating that insubstantial or inconsequential modifications or alterations of the subject matter described and claimed are considered to be within the scope of the invention as recited in the appended claims.

It should be noted that the term “exemplary” as used herein to describe various embodiments is intended to indicate that such embodiments are possible examples, representations, and/or illustrations of possible embodiments (and such term is not intended to connote that such embodiments are necessarily extraordinary or superlative examples).

The terms “coupled,” “connected,” and the like, as used herein, mean the joining of two members directly or indirectly to one another. Such joining may be stationary (e.g., permanent) or moveable (e.g., removable, releasable, etc.). Such joining may be achieved with the two members or the two members and any additional intermediate members being integrally formed as a single unitary body with one another or with the two members or the two members and any additional intermediate members being attached to one another.

References herein to the positions of elements (e.g., “top,” “bottom,” “above,” “below,” etc.) are merely used to describe the orientation of various elements in the figures. It should be noted that the orientation of various elements may differ according to other exemplary embodiments, and that such variations are intended to be encompassed by the present disclosure.

Also, the term “or” is used in its inclusive sense (and not in its exclusive sense) so that when used, for example, to connect a list of elements, the term “or” means one, some, or all of the elements in the list. Conjunctive language such as the phrase “at least one of X, Y, and Z,” unless specifically stated otherwise, is otherwise understood with the context as used in general to convey that an item, term, etc. may be either X, Y, Z, X and Y, X and Z, Y and Z, or X, Y, and Z (i.e., any combination of X, Y, and Z). Thus, such conjunctive language is not generally intended to imply that certain embodiments require at least one of X, at least one of Y, and at least one of Z to each be present, unless otherwise indicated.

It is important to note that the construction and arrangement of the elements of the systems and methods as shown in the exemplary embodiments are illustrative only. Although only a few embodiments of the present disclosure have been described in detail, those skilled in the art who review this disclosure will readily appreciate that many modifications are possible (e.g., variations in sizes, dimensions, structures, shapes and proportions of the various elements, values of parameters, mounting arrangements, use of materials, colors, orientations, etc.) without materially departing from the novel teachings and advantages of the subject matter recited. For example, elements shown as integrally formed may be constructed of multiple parts or elements. It should be noted that the elements and/or assemblies of the components described herein may be constructed from any of a wide variety of materials that provide sufficient strength or durability, in any of a wide variety of colors, textures, and combinations. Accordingly, all such modifications are intended to be included within the scope of the present inventions. Other substitutions, modifications, changes, and omissions may be made in the design, operating conditions, and arrangement of the preferred and other exemplary embodiments without departing from scope of the present disclosure or from the spirit of the appended claims.

1. A system for monitoring and tracking swelling of a patient's limb, the system comprising:
a dressing positioned on a patient's limb over a wound, the dressing comprising a pattern indicator;
an imaging device configured to obtain an image of the dressing and the pattern indicator; and
processing circuitry configured to:
obtain an initial image of the pattern indicator;
obtain one or more subsequent images of the pattern indicator at later times; and
determine a swelling change of the patient's limb using the initial image and the one or more subsequent images.

2. The system of claim 1, wherein the processing circuitry and the imaging device are components of a smartphone.

3. The system of claim 1, wherein the processing circuitry is configured to:
perform an image analysis technique to determine a baseline dimension of the pattern indicator using the initial image of the pattern indicator;
perform the image analysis technique to determine one or more subsequent dimensions of the pattern indicator using the one or more subsequent images of the pattern indicator obtained at later times; and
determine relative increase or decrease amounts between the baseline dimension of the pattern indicator and the one or more subsequent dimensions of the pattern indicator.

4. The system of claim 3, wherein the processing circuitry is configured to:
determine a relative increase or decrease in at least one of a circumference, diameter, or volume of the patient's limb using the relative increase or decrease amounts.

5. The system of claim 1, wherein the pattern indicator comprises:
a grid configured to expand or contract with swelling increases or decreases of the patient's limb; and
a reference indicator fixedly coupled with the grid, wherein the reference indicator is a rigid member that does not expand or contract with swelling increases or decreases of the patient's limb.

6. The system of claim 5, wherein the processing circuitry is configured to calibrate any of the initial image or the one or more subsequent images using image data of the reference indicator to account for a relative orientation or position between the imaging device and the pattern indicator.

7. The system of claim 1, wherein the processing circuitry is configured to:
generate progress data of a plurality of values of the swelling change of the patient's limb using the initial image and a plurality of subsequent images;
generate a progress report using the progress data; and
provide the progress report to at least one of the patient via a display screen or a healthcare professional via a remote system.

8. The system of claim 7, wherein the progress report comprises at least one of:
a current value of a circumference, diameter, or volume of the patient's limb;
a current value of a dimension of the pattern indicator;
historical values of the circumference, diameter, or volume of the patient's limb;
historical values of the dimension of the pattern indicator; or
a graph showing swelling changes of the patient's limb over time.

9. The system of claim 1, wherein the system further comprises a display screen, wherein the processing circuitry is configured to periodically prompt the patient to capture a new image of the pattern indicator via the display screen.

10. The system of claim 1, wherein the processing circuitry is configured to alert the patient or a healthcare professional in response to the swelling increasing by a predetermined amount or more.

11. (canceled)

12. A controller for monitoring and tracking swelling of a patient's limb, the controller comprising processing circuitry configured to:
obtain an initial image of a pattern indicator positioned at the patient's limb using an imaging device;
obtain one or more subsequent images of the pattern indicator at later times using the imaging device;
determine a swelling change of the patient's limb using the initial image and the one or more subsequent images; and
provide a report to at least one of the patient or a healthcare provider including the swelling change of the patient's limb.

13. The controller of claim 12, wherein the controller is a patient's smartphone with a mobile application installed on the processing circuitry, wherein the processing circuitry and the imaging device are components of the patient's smartphone.

14. The controller of claim 12, wherein the processing circuitry is configured to:
perform an image analysis technique to determine a baseline dimension of the pattern indicator using the initial image of the pattern indicator;
perform the image analysis technique to determine one or more subsequent dimensions of the pattern indicator using the one or more subsequent images of the pattern indicator obtained at later times; and
determine relative increase or decrease amounts between the baseline dimension of the pattern indicator and the one or more subsequent dimensions of the pattern indicator.

15. The controller of claim 14, wherein the processing circuitry is configured to: determine a relative increase or decrease in at least one of a circumference, diameter, or volume of the patient's limb using the relative increase or decrease amounts.

16. The controller of claim 12, wherein the pattern indicator comprises:
a grid configured to expand or contract with swelling increases or decreases of the patient's limb; and
a reference indicator fixedly coupled with the grid, wherein the reference indicator is a rigid member that does not expand or contract with swelling increases or decreases of the patient's limb.

17. The controller of claim 16, wherein the processing circuitry is configured to calibrate any of the initial image or the one or more subsequent images using image data of the reference indicator to account for a relative orientation or position between the imaging device and the pattern indicator.

18. The controller of claim 12, wherein the processing circuitry is configured to:
generate progress data of a plurality of values of the swelling change of the patient's limb using the initial image and a plurality of subsequent images;
generate a progress report using the progress data; and
provide the progress report to at least one of the patient via a display screen or a healthcare professional via a remote system.

19. The controller of claim 18, wherein the progress report comprises at least one of:
a current value of a circumference, diameter, or volume of the patient's limb;
a current value of a dimension of the pattern indicator;
historical values of the circumference, diameter, or volume of the patient's limb;
historical values of the dimension of the pattern indicator; or
a graph showing swelling changes of the patient's limb over time.

20. The controller of claim 12, wherein the processing circuitry is configured to periodically prompt the patient to capture a new image of the pattern indicator via a display screen.

21. (canceled)

22. A method for monitoring and tracking swelling of a patient's limb, the method comprising:
obtaining an initial image of a pattern indicator positioned at the patient's limb using an imaging device;
obtaining one or more subsequent images of the pattern indicator at later times using the imaging device;
determining a swelling change of the patient's limb using the initial image and the one or more subsequent images; and
providing a report to at least one of the patient or a healthcare provider including the swelling change of the patient's limb.

23.-30. (canceled)
